(19) United States
(12) Patent Application Publication
(10) Pub. No.: US 2024/0130715 A1
(43) Pub. Date: Apr. 25, 2024

(54) METHOD AND DEVICE FOR DETECTING MEDICAL INDICES IN MEDICAL IMAGES

(71) Applicant: ONTACT HEALTH CO., LTD., Seoul (KR)

(72) Inventors: Se Keun KIM, Goyang-si, Gyeonggi-do (KR); Hack Joon SHIM, Seoul (KR); Young Taek HONG, Seoul (KR); Seong Min HA, Seoul (KR); Shin Hee MAENG, Seoul (KR)

(21) Appl. No.: 18/548,074

(22) PCT Filed: Feb. 24, 2022
(86) PCT No.: PCT/KR2022/002740

§ 371 Date: Aug. 25, 2023

(30) Foreign Application Priority Data
Feb. 26, 2021 (KR) 10-2021-0026361
Oct. 18, 2021 (KR) 10-2021-0139303

(51) Int. Cl. A61B 8/08; G06T 7/00; G06T 7/11

(52) U.S. Cl. CPC A61B 8/5223; A61B 8/0883; G06T 7/0014; G06T 7/11; G06T 2207/30048

(57) ABSTRACT

In order to detect medical indices in a medical image, a method of obtaining information from a medical image may include performing segmentation on regions of a heart in the medical image, generating at least one reference line based on the segmentation and determining at least one medical index based on the at least one reference line.

### TECHNICAL FIELD

The present invention relates to processing of medical images and, more particularly, to a method and device for detecting medical indices in medical images.

### BACKGROUND ART

Diseases are a condition that causes a disorder and thus impedes the normal function of a human mind or body, and depending on the seriousness of diseases, man undergoes sufferings and even end their lives. Accordingly, over the course of human history, a variety of social systems and technologies have been developed to diagnose, treat and even prevent diseases. For diagnosis and treatment of diseases, various tools and methods have been devised along with impressive technical advances, but the final judgments are still dependent on doctors.

Meanwhile, the recent advancement of artificial intelligence (AI) technology is so remarkable as to draw attention from various fields. In particular, massive accumulations of medical data and the image-centered diagnostic data environment encourage various attempts and studies to graft AI algorithms onto medicine. Specifically, various studies are using AI algorithms to provide solutions to the diagnosis and prediction of diseases and other tasks that still depend on clinical judgments. In addition, various studies are being conducted to solve the task of processing and analyzing medical data as an intermediate process for diagnosis using artificial intelligence algorithms.

### Technical Problem

An object of the present invention is to provide a method and device for effectively obtaining information from a medical image using an artificial intelligence (AI) algorithm.

An object of the present invention is to provide a method and device for automatically detecting medical indices from a medical image.

An object of the present invention is to provide a method and device for detecting medical indices based on segmentation of a medical image.

It is to be understood that the technical objects to be achieved by the present invention are not limited to the aforementioned technical objects, and other technical objects not mentioned herein will be apparent to those of ordinary skill in the art to which the present invention pertains from the following description.

### Technical Solution

A method of obtaining information from a medical image according to an embodiment of the present invention may include performing segmentation on regions of a heart in the medical image, generating at least one reference line based on the segmentation and determining at least one medical index based on the at least one reference line.

According to an embodiment of the present invention, the at least one medical index may include at least one of a length of the at least one reference line or a value determined based on the length of the at least one reference line.

According to an embodiment of the present invention, the method may further include generating at least one reference point based on the segmentation.

According to an embodiment of the present invention, the at least one reference point may be generated based on at least one center point of the regions.

According to an embodiment of the present invention, the regions may include at least one of a first region, a second region, a third region, a fourth region, a fifth region or a sixth region.

According to an embodiment of the present invention, the first region may be located between the second region and the third region, the second region may be located between the fourth region and the first region, the fourth region may be located at the top, the fifth region may be adjacent to a right side of the first region, or the sixth region may be adjacent to the right side of the first region and be located below the fifth region.

According to an embodiment of the present invention, the generating the at least one reference line may include identifying a transversal line passing through a reference point of the first region and a center of a left boundary line of the first region and crossing the first region, identifying a first orthogonal line passing through the reference line and orthogonal to the transversal line and generating a first reference line to include at least a part of the first orthogonal line.

According to an embodiment of the present invention, the generating the at least one reference line may include identifying a second orthogonal line passing through a point of contact between the first reference line and a segmentation contour of the second region and orthogonal to a long axis of the second region and generating a second reference line to include at least a part of the second orthogonal line.

According to an embodiment of the present invention, the generating the at least one reference line may include identifying a third orthogonal line passing through a point of contact between the first reference line and a segmentation contour of the third region and orthogonal to a long axis of the third region and generating a third reference line to include at least a part of the third orthogonal line.

According to an embodiment of the present invention, the generating the at least one reference line may include identifying a center line of a short axis of a region corresponding to the second region, identifying a first vertical line perpendicular to a boundary line of the second region at a point where the center line and the boundary line of the region meet, and generating a first reference line to include at least a part of the first vertical line, and the first reference line may be used to measure a diameter of the first region.

According to an embodiment of the present invention, the generating the at least one reference line may include identifying a second vertical line perpendicular to a boundary line of the third region at a point where the first reference line and the boundary line of the third region meet and generating a second reference line to include at least a part of the second vertical line, and the second reference line may be used to measure a thickness of the third region.

According to an embodiment of the present invention, the generating the at least one reference line may include identifying a closest point closest to the fourth region among points on a boundary line of the fifth region, identifying a parallel line including the closest point and parallel to a junction of the first region and the fifth region, and generating a third reference line to include at least a part of the parallel line.

According to an embodiment of the present invention, the generating the at least one reference line may include identifying a sinus point on a boundary line of the fifth region, identifying a vertical line including a point closest to the sinus point on a boundary line of a region corresponding to the sixth region, parallel to a vertical axis of the medial image and penetrating the inside of a region corresponding to the sixth region and generating a fourth reference line to include at least a part of the vertical line.

According to an embodiment of the present invention, the generating the at least one reference line may include identifying a sinus point on a boundary line of the fifth region, identifying a vertical line including a point closest to the sinus point on a boundary line of a region corresponding to the sixth region, perpendicular to a long axis of a region corresponding to the sixth region and penetrating the inside of the region corresponding to the sixth region, and determining a fourth reference line to include at least a part of the vertical line.

According to an embodiment of the present invention, the generating the at least one reference line may include identifying a vertical line including one point on a junction of the fifth region and the first region and perpendicular to a center line of a long axis of the fourth region and generating a fifth reference line to include at least a part of the vertical line.

According to an embodiment of the present invention, the generating the at least one reference line may include identifying a vertical line including one point on a junction of the fifth region and the first region and perpendicular to a vertical axis of the medical image and generating a fifth reference line to include at least a part of the vertical line.

According to an embodiment of the present invention, the regions may include at least one of right ventricle (RV), interventricular septum (IVS), aorta, left ventricle (LV), LV posterior wall (LVPW) or left atrium (LA).

A device for obtaining information from a medical image according to an embodiment of the present invention may include a storage unit configured to store a set of commands for operation of the device and at least one processor connected to the storage unit. The at least one processor may perform segmentation on regions of a heart in the medical image, generate at least one reference line based on the segmentation, and determine at least one medical index based on the at least one reference line.

A program stored on a medium according to an embodiment of the present invention may implement the above-described method when operated by a processor.

The features briefly summarized above for the present invention are only illustrative aspects of the detailed description of the invention that follows, but do not limit the scope of the present invention.

### Advantageous Effects

According to the present invention, it is possible to efficiently obtain information from medical images using an artificial intelligence (AI) algorithm.

It is to be understood that effects to be obtained by the present invention are not limited to the aforementioned effects, and other effects not mentioned herein will be apparent to those of ordinary skill in the art to which the present invention pertains from the following description.

### DESCRIPTION OF DRAWINGS

FIG. 1 illustrates a system according to an embodiment of the present invention.

FIG. 2 illustrates a structure of a device according to an embodiment of the present invention.

FIG. 3 illustrates an example of a perceptron constituting an artificial intelligence model applicable to the present invention.

FIG. 4 illustrates an example of an artificial neural network constituting an artificial intelligence model applicable to the present invention.

FIG. 5 illustrates a mechanism for determining a landmark detection method using a regression model according to an embodiment of the present invention.

FIGS. 6A, 6B, and 6C illustrate examples of detection results for each landmark detection method.

FIG. 7 illustrates an example of a procedure for detecting medical indices based on segmentation according to an embodiment of the present invention.

FIG. 8 illustrates an example of a segmentation result of a medical image according to an embodiment of the present invention.

FIG. 9 illustrates an example of a procedure for determining a reference line according to an embodiment of the present invention.

FIG. 10 illustrates another example of a procedure for determining a reference line according to an embodiment of the present invention.

FIG. 11 illustrates another example of a procedure for determining a reference line according to an embodiment of the present invention.

FIG. 12 illustrates another example of a procedure for determining a reference line according to an embodiment of the present invention.

FIG. 13A illustrates an example of reference lines determined in an image in an end-diastole state according to an embodiment of the present invention.

FIG. 13B illustrates an example of reference lines determined in an image in an end-systole state according to an embodiment of the present invention.

FIG. 14 illustrates an example of reference points and reference lines determined based on a segmentation result according to an embodiment of the present invention.

FIGS. 15A and 15B illustrate an example of a long short-term memory (LSTM) network applicable to the present invention.

FIG. 16 illustrates an example of a training procedure for segmentation according to an embodiment of the present invention.

FIG. 17 illustrates a heart image and a propagated heart image according to an embodiment of the present invention.

FIG. 18 illustrates an example of a segmentation procedure according to an embodiment of the present invention.

FIGS. 19A, 19B, and 19C illustrate specific examples of data augmentation according to an embodiment of the present invention.

FIG. 20 illustrates a specific implementation example of an artificial intelligence model for segmentation according to an embodiment of the present invention.

FIG. 21 illustrates another specific implementation example of an artificial intelligence model for segmentation according to an embodiment of the present invention.

### DETAILED DESCRIPTION

Hereinafter, embodiments of the present invention will be described in detail with reference to the accompanying drawings, which will be easily implemented by those skilled in the art. However, the present invention may be embodied in many different forms and is not limited to the embodiments described herein.

In the following description of the exemplary embodiments of the present invention, a detailed description of known functions and configurations incorporated herein will be omitted when it may make the subject matter of the present invention rather unclear. In addition, parts not related to the description of the present invention in the drawings are omitted, and like parts are denoted by similar reference numerals.

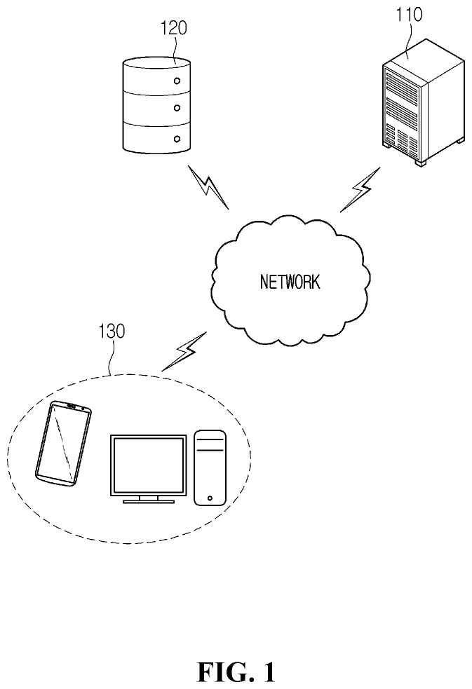

FIG. 1 illustrates a system according to an embodiment of the present invention.

Referring to FIG. 1, a system may include a service server 110, a data server 120, and at least one client device 130.

The service server 110 provides a service based on an artificial intelligence model. That is, the service server 110 performs a learning and prediction operation by using the artificial intelligence model. The service server 110 may perform communication with the data server 120 or the at least one client device 130 via a network. For example, the service server 110 may receive learning data for training the artificial intelligence model from the data server 120 and perform training. The service server 110 may receive data necessary for a learning and prediction operation from the at least one client device 130. In addition, the service server 110 may transmit information on a prediction result to the at least one client device 130.

The data server 120 provides learning data for training of an artificial intelligence model stored in the service server 110. According to various embodiments, the data server 120 may provide public data accessible to anyone or data requiring permission. When necessary, learning data may be preprocessed by the data server 120 or the service server 120. According to another embodiment, the data server 120 may be omitted. In this case, the service server 110 may use an artificial intelligence model that is externally trained, or learning data may be provided offline to the service server 110.

The at least one client device 130 transmits and receives data associated with an artificial intelligence model, which is managed by the service server 110, to and from the service server 110, respectively. The at least one client device 130 may be an equipment used by a user, transmits information input by the user to the service server 110, and store or provide (e.g., mark) information received from the service server 110 to the user. According to a situation, a prediction operation is performed based on data transmitted from any one client, and information associated with a result of prediction may be provided to another client. The at least one client device 130 may be a computing device with various forms like a desktop computer, a laptop computer, a smartphone, a tablet PC, and a wearable device.

Although not illustrated in FIG. 1, the system may further include a management device for managing the service server 110. Being a device used by a subject that manages a service, the management device monitors a state of the service server 110 or controls a setting of the service server 110. The management device may access the service server 110 via a network or be directly connected with the service server 110 through a cable connection. According to a control of the management device, the service server 110 may set a parameter for operation.

As described with reference to FIG. 1, the service server 110, the data server 120, the at least one client device 130, and a management device may be connected via a network and interact with each other. Herein, the network may include at least one of a wired network and a wireless network and consist of any one of a cellular network, a short-range network, and a wide area network or a combination of two or more thereof. For example, the network may be embodied based on at least one of a local area network (LAN), a wireless LAN (WLAN), Bluetooth, LTE (long term evolution), LTE-A (LTE-advanced), and 5G (5th generation).

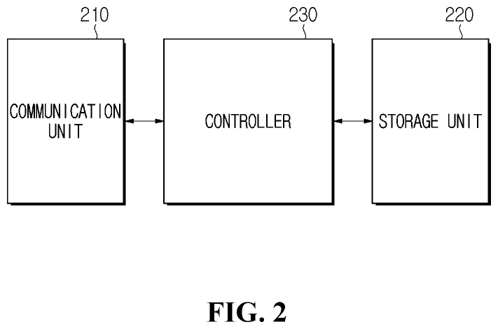

FIG. 2 illustrates a structure of a device according to an embodiment of the present invention. The structure exemplified in FIG. 2 may be understood as a structure of the service server 110, the data server 120, and the at least client device 130 of FIG. 1.

Referring to FIG. 2, the device includes a communication unit 210, a storage unit 220, and a controller 230.

The communication unit 210 accesses a network and performs a function for communicating with another device. The communication unit 210 supports at least one of wired communication and wireless communication. For communication, the communication unit 210 may include at least one of a radio frequency (RF) processing circuit and a digital data processing circuit. According to a case, the communication unit 210 may be understood as a component including a terminal for connecting a cable. Since the communication unit 210 is a component for transmitting and receiving data and a signal, the communication unit 210 may be referred to as ‘transceiver’.

The storage unit 220 stores data, a program, a micro code, a set of instructions, and an application, which are necessary to operate a device. The storage unit 220 may be embodied as a temporary or non-temporary storing medium. In addition, the storage unit 210 may be embodied in a fixed form in a device or in a separable form. For example, the storage unit 220 may be embodied in at least one of a NAND flash memory such as a compact flash (CF) card, a secure digital (SD) card, a memory stick, a solid-state drive (SSD) and a micro SD card and a magnetic computer memory device like a hard disk drive (HDD).

The controller 230 controls an overall operation of a device. To this end, the controller 230 may include at least one processor and at least one microprocessor. The controller 230 may execute a program stored in the storage unit 220 and access a network via the communication unit 210. Particularly, the controller 230 may execute algorithms according to various embodiments described below and control a device to operate according to the embodiments described below.

Based on a structure described with reference to FIG. 1 and FIG. 2, a service based on an artificial intelligence algorithm may be provided according to various embodiments of the present invention. Herein, an artificial intelligence model consisting of an artificial neural network may be used to implement an artificial intelligence algorithm. The concepts of perceptron, which is a constituent unit of an artificial neural network, and the artificial neural network are as follows.

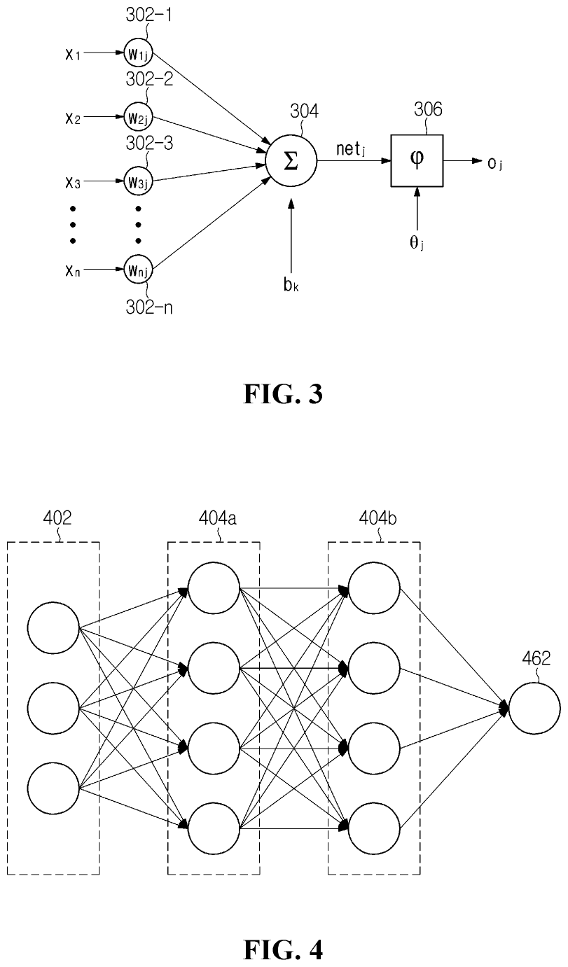

Being modeled after neurons of a living thing, perceptrons have a structure of outputting a single signal from a plurality of input signals. FIG. 3 illustrates an example of a perceptron constituting an artificial intelligence model applicable to the present invention. Referring to FIG. 3, a perceptron multiplies each input value (e.g., x₁, x₂, x₃, . . . , xₙ) by weights 302-1 to 302-n (e.g., w₁ⱼ, w₂ⱼ, w₃ⱼ, . . . , wₙⱼ) and then adds up the weighted input values by using a transfer function 304. During the adding-up process, a bias value (e.g., bₖ) may be added. A perceptron generates an output value (e.g., oⱼ) by applying an activation function 406 for a net input value (e.g., netⱼ) that is an output of the transfer function 304. According to a case, the activation function 406 may operate based on a threshold (e.g., θⱼ). The activation function may be defined in various ways. A step function, a sigmoid, a Relu, and a Tanh may be used as an activation function, and the present invention is not limited thereto.

As illustrated in FIG. 3, an artificial neural network may be designed when perceptrons are arranged to form a layer. FIG. 4 illustrates an example of an artificial neural network constituting an artificial intelligence model applicable to the present invention. In FIG. 4, each node represented as a circle may be understood as a perceptron of FIG. 3. Referring to FIG. 4, an artificial neural network includes an input layer 402, a plurality of hidden layers 404a and 404b, and an output layer 406.

In case prediction is performed, when input data is provided to each node of the input layer 402, the input data is forward propagated to the output layer 406 through the input layer 402, weight application by perceptrons constituting the hidden layers 404a and 404b, a transfer function operation, and an activation function operation. On the other hand, in case training is performed, an error may be calculated through backward propagation from the output layer 406 to the input layer 402, and weights defined in each perceptron may be updated according to the calculated error.

The present invention proposes a technique for detecting medical indices from medical images. Specifically, the present invention proposes a technique for deriving clinically important indices by more robustly segmenting multiple structures (e.g., left atrium, left ventricle, right atrium, right ventricle, inner wall, etc.) in a 2D ultrasonic image used to evaluate or diagnose a patient's heart shape, structure, or function) and detecting major landmarks (e.g., Apex, Annulus, etc.).

Echocardiography is a primary and essential examination that is used daily, and it is non-invasive and may be performed at a low cost. Echocardiography allows structural and functional evaluation of the heart, and secondary examinations may be subsequently performed if additional examinations are required. Due to the above features, constructing data from echocardiography images is relatively easier than other medical equipment-based methods. However, since the manpower cost and time cost of medical experts generated during the analysis process are high, echocardiography images are data that have the potential to further increase usefulness.

In the case of echocardiographic data, a subdivided name is determined based on the location where the heart was imaged according to the recommended standards of the American Heart Association, and the echocardiography analysis method is also different according to the subdivided name. Therefore, an expert in echocardiography analysis is required to quantify echocardiographic data. Recently, in order to solve this problem, studies for automating echocardiography analysis have been conducted, and studies for segmenting the structural location of the left ventricle using artificial intelligence techniques have been reported.

Although a number of techniques for segmenting specific cardiac structures using artificial intelligence have been introduced, techniques for detecting landmarks that are actually clinically meaningful are far fewer than segmentation techniques, and most of them are performed in specific image views (e.g., A2CH, A3CH, A4CH, PSAX, PLAX (parasternal long axis), etc.). For this reason, even if they have the same structure, the image views that look different from each other cannot be considered, which may cause performance degradation.

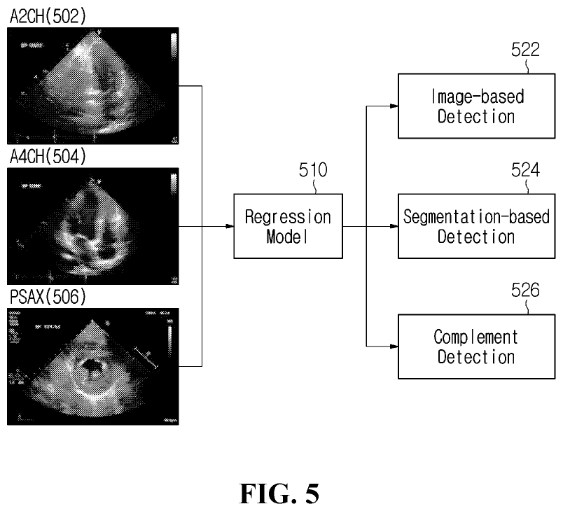

Even if the same heart structure is included in the image, it has different characteristics depending on the image view. For example, A2CH includes only the left atrium and left ventricle, and A4CH includes not only left atrium and left ventricle but also right atrium and right ventricle. As such, A2CH and A4CH represent the same heart structure, but, for more robust landmark detection from each of the views representing different organs, as shown in FIG. 5, it may be determined whether landmarks are detected from the image itself or from a result of segmentation for a given view through a regression model.

FIG. 5 illustrates a mechanism for determining a landmark detection method using a regression model according to an embodiment of the present invention. Referring to FIG. 5, images captured from views, such as A2CH 502, A4CH 504, and PSAX 506, are input to a regression model 510. One of image-based detection 522, segmentation-based detection 524, and complement detection 526 may be selected by the regression model 510. As described above, when an image of a specific view is provided as input of the regression model 510, which view is provided may be predicted, and through this, which approach is used may be determined. Since a specific structure visible to the naked eye is different for each view, one of image-based detection 522, segmentation-based detection 524, and complement detection 526 is used to process images more efficiently.

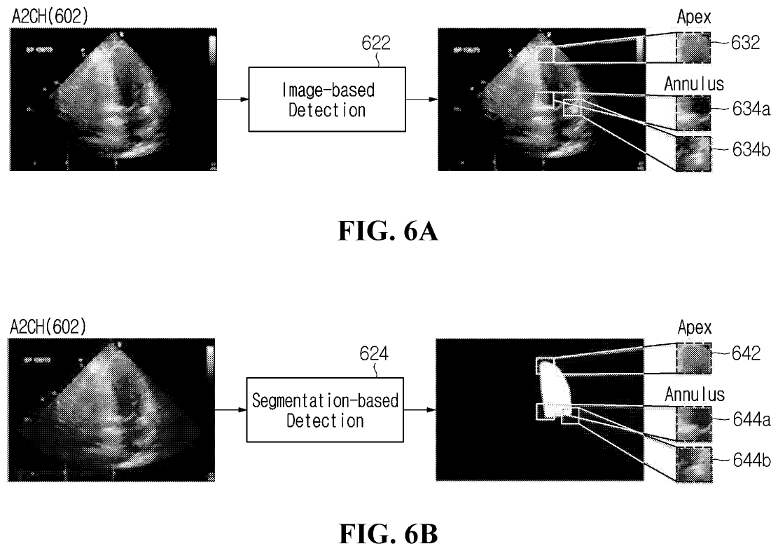

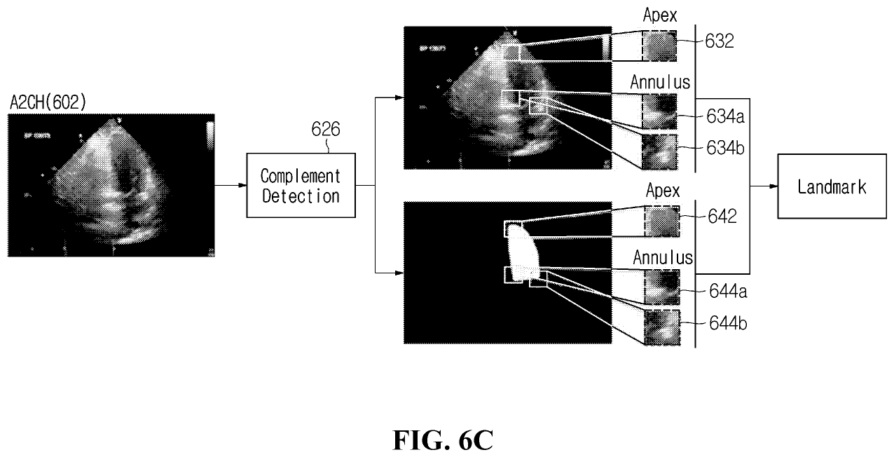

FIGS. 6A, 6B, and 6C illustrate examples of detection results for each landmark detection method. Referring to FIG. 6A, A2CH 602 may be analyzed by image-based detection 622. In the case of image-based detection 622, landmarks (e.g., Apex 632 and Annulus 634a, 634b) may be detected using only ultrasonic images. The image-based detection 622 has the advantage of being able to detect landmarks more quickly, because landmarks are detected only from images without performing segmentation.

Referring to FIG. 6B, A2CH 602 may be analyzed by segmentation-based detection 624. In the case of the segmentation-based detection 624, after segmentation is performed on a ultrasonic image, a landmark is detected from a segmentation result. Therefore, it takes relatively much time compared to the image-based detection 622, but the time required has been greatly reduced through recent artificial intelligence. In addition, since the artificial intelligence algorithm derives image segmentation results that are acceptable to clinicians, landmarks (e.g., Apex 642, Annulus 644a, 644b) may be detected with relatively higher accuracy than landmark detection based only on a single image, such as the image-based detection 622.

Referring to FIG. 6C, A2CH 602 may be analyzed by complement detection 626. The complement detection 626 may be used when it is difficult to detect landmarks using only one of the image-based detection 622 and the segmentation-based detection 624. In the complement detection 626, probabilities determined by the image-based detection 622 (e.g., Apex 632, Annulus 634a, 634b) and probabilities determined by the segmentation-based detection 624 (e.g., Apex 642, Annulus 644a, 644b) may be refined and complemented by a Bayesian method, and landmarks may be detected.

Guidelines for measuring various medical indices in the echocardiography images are defined by the American Society of Echocardiography (ASE). In order to measure medical indices according to the guidelines of the ASE, clinically specific landmarks defined in the ASE are needed. At this time, in order to detect specific landmarks in the patient's echocardiography images that needs to be analyzed, human intervention is unavoidable, which takes time and money. Accordingly, the present invention proposes a technique for measuring medical indices without human intervention. Through the technology proposed in the present invention, specific landmarks may be automatically detected in the patient's echocardiography images that needs to be analyzed, and it is expected that the required time and cost will be greatly reduced compared to the conventional method.

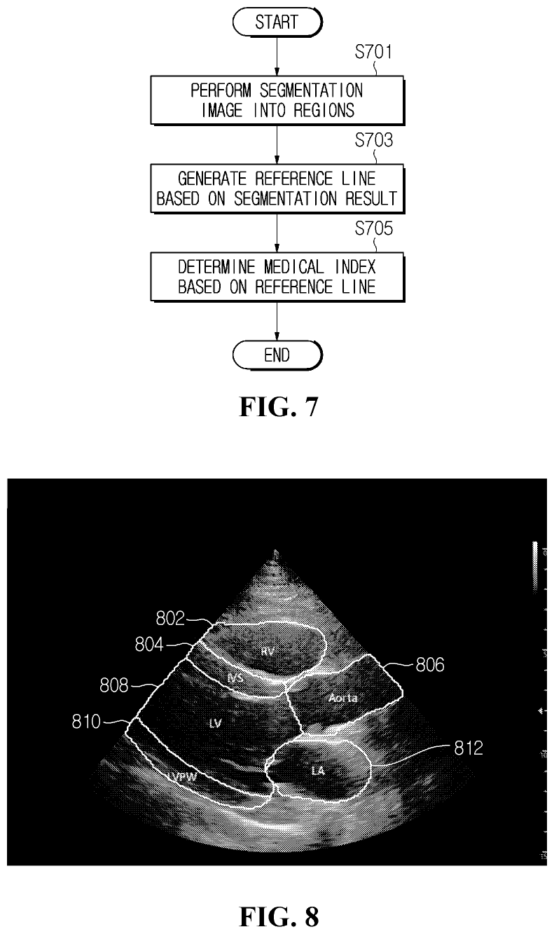

FIG. 7 illustrates an example of a procedure for detecting medical indices based on segmentation according to an embodiment of the present invention. FIG. 7 illustrates a method of operating a device having computing capability (e.g., the service server 110 of FIG. 1).

Referring to FIG. 7, in step S701, the device performs segmentation on an image into regions. The device is designed for segmentation and may perform segmentation using a trained artificial intelligence model. For example, the image may be an echocardiography image and may be an image of a PLAX view. The regions refer to spaces, muscles, and blood vessels that constitute the heart. For example, as shown in FIG. 8, by segmentation, right ventricle (RV) 802, interventricular septum (IVS) 804, aorta 806, left ventricle (LV) 808, LVPW (LV posterior wall 810 and a left atrium (LA) 812 may be identified.

In step S703, the device generates at least one reference line based on the segmentation result. The reference line may be generated based on the regions obtained by segmentation. For example, the reference line may be determined based on a relative position between the regions, a point based on a positional relationship between the regions, a boundary line of each region, and the like.

In step S705, the device determines at least one medical index based on the at least one reference line. For example, the at least one medical index may include a length of at least one generated reference line, a value determined based on the at least one reference line, and the like.

Although not shown in FIG. 7, at least one medical index determined based on at least one reference line may be provided to a user. For example, the device may display at least one medical index through a display device. In this case, at least one medical index may be displayed to overlap a medical image or displayed through a separate interface. When it is displayed so as to overlap the medical image, each medical index may be disposed in a region within a threshold distance from the reference line. Additionally, an indicator (e.g., a connection line, etc.) representing a relationship between each medical index and the corresponding reference line may be displayed together. As another example, the device may transmit data representing at least one medical index to another device through a network.

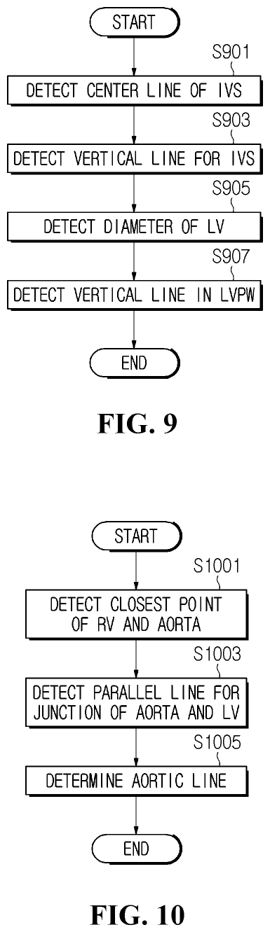

FIG. 9 illustrates an example of a procedure for determining a reference line according to an embodiment of the present invention. FIG. 9 illustrates a method of operating a device having computing capability (e.g., the service server 110 of FIG. 1).

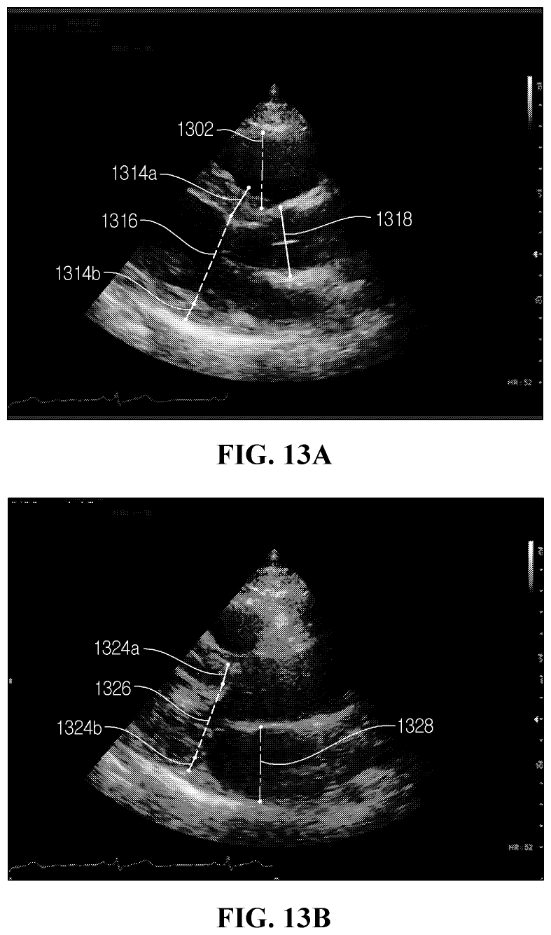

Referring to FIG. 9, in step S901, the device detects a center line of the IVS. The device detects a line crossing a short axis of a region corresponding to the IVS. For example, the center line of the IVS is line 1314a in FIG. 13A or line 1314b in FIG. 13B.

In step S903, the device detects a vertical line for the IVS. The device detects a line perpendicular to the boundary line of the IVS at a point where the center line of the IVS and the boundary line of the IVS meet. In other words, the device detects a line perpendicular to the boundary line of the IVS at the point where the center line detected in step S901 and the boundary line of the region corresponding to the IVS meet. Here, the vertical line may penetrate the inside of the region corresponding to the LV. For example, the vertical line for IVS is line 1316 in FIG. 13A or line 1326 in FIG. 13B.

In step S905, the device detects a diameter of the LV. That is, the device may detect the diameter of the LV by measuring the length of the vertical line detected in step S903. That is, the vertical line detected in step S903 is one of the medical indices and may be used to measure the diameter of the LV.

In step S907, the device detects a vertical line in the LVPW. That is, the device detects a line perpendicular to the boundary line at the point where the vertical line detected in step S903 and the boundary line of the LVPW meet. The vertical line may penetrate the inside of the region corresponding to the LVPW. For example, the vertical line within the LVPW is line 1314b in FIG. 13A or line 1324b in FIG. 13B. Here, the length of the vertical line is one of the medical indices to be measured and may be used to measure the thickness of the LVPW.

FIG. 10 illustrates another example of a procedure for determining a reference line according to an embodiment of the present invention. FIG. 10 illustrates a method of operating a device having computing capability (e.g., the service server 110 of FIG. 1).

Referring to FIG. 10, in step S1001, the device detects the closest point of the RV and the aorta. For example, the closest point may be a point closest to the RV among points on the boundary line of the aorta. Here, the closest point may be a sinus point. The sinus point refers to a point having predefined characteristics among points on the boundary line of the aorta. For example, the sinus point may refer to a low point of a downwardly convex part at the lower end of the boundary line of the aorta determined in the echocardiography image of the PLAX view.

In step S1003, the device detects a parallel line for a junction of the aorta and the LV. The junction of the aorta and the LV refers to a line or at least one point where the boundary line of the aorta and the boundary line of the LV obtained by segmentation overlap. The parallel line is an overlapping line, that is, a line parallel to the line corresponding to the junction and including the closest point detected in step S1001. That is, the parallel line starts from the closest point and passes through the inside of the region corresponding to the aorta. For example, the parallel line may be line 1318 in FIG. 13A.

In step S1005, the device determines an aortic line. Here, the aortic line means the parallel line detected in step S1003. The length of the aortic line may be one of the medical indices to be measured.

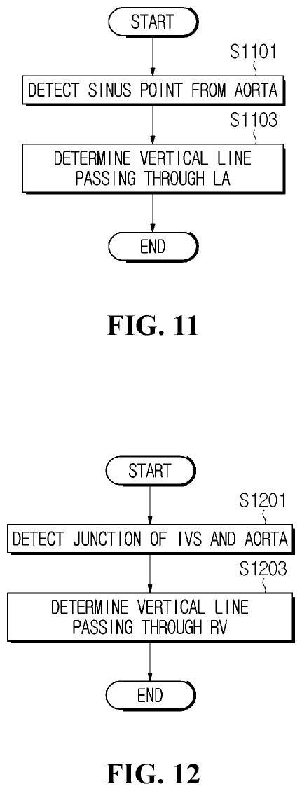

FIG. 11 illustrates another example of a procedure for determining a reference line according to an embodiment of the present invention. FIG. 11 illustrates a method of operating a device having computing capability (e.g., the service server 110 of FIG. 1).

In step S1101, the device detects a sinus point from the aorta. That is, the device detects a sinus point on the boundary line of the aorta. The sinus point refers to a point having predefined features among points on the boundary line of the aorta. For example, the sinus point may refer to a high point or a low point of a convex part upward from the upper end or downward from the lower end of the boundary line of the aorta determined in the echocardiography image of the PLAX view.

In step S1103, the device determines a vertical line passing through the LA. One end of the vertical line coincides with the sinus point detected in step S1101. According to an embodiment, the vertical line may be a line perpendicular to the LA at the sinus point and penetrating the inside of a region corresponding to the LA. According to another embodiment, the vertical line may be a line including a point closest to the sinus point on the boundary line of the region corresponding to the LA, perpendicular to the center line of the long axis of the LA, and penetrating the inside of the region corresponding to the LA. According to another embodiment, the vertical line may be a line including a point closest to the sinus point on the boundary line of the region corresponding to the LA, parallel to the vertical axis of the medical image, and penetrating the inside of the region corresponding to the LA. For example, the vertical line may be line 1328 in FIG. 13B. The vertical line length may be one of the medical indices to be measured.

FIG. 12 illustrates another example of a procedure for determining a reference line according to an embodiment of the present invention. FIG. 12 illustrates a method of operating a device having computing capability (e.g., the service server 110 of FIG. 1).

In step S1201, the device detects a junction of the IVS and the aorta. The junction of the IVS and the aorta refers to a line or at least one point where the boundary line of the IVS and the boundary line of the aorta obtained by segmentation overlap.

In step S1203, the device determines a vertical line passing through the RV. One end of the vertical line coincides with a point on the junction detected in step S1201. According to an embodiment, the vertical line may be a line parallel to the vertical axis of the image. According to another embodiment, the vertical line may be a line perpendicular to the center line of the long axis of the RV. For example, the vertical line may be line 1312 in FIG. 13A. The vertical line length may be one of the medical indices to be measured.

In the embodiment described with reference to FIG. 12, a junction of the IVS and the aorta is used. According to another embodiment, instead of the junction of the IVS and the aorta, a point where a line extending the junction of the LV and the aorta and the boundary of the region corresponding to the RV meet may be used.

According to various embodiments described above, at least one medical index may be determined based on the segmentation result of a medical image. For example, examples of medical indices that may be measured from echocardiography of the PLAX view are shown in [Table 1] below.

TABLE 1

| | Pearson |
|---|---|
| | correlation |
| Measurement | coefficient (r) |
| LV septum diameter at the diastole (unit: mm) | 0.85 |
| LV septum diameter at the systole (unit: mm) | 0.84 |
| LV internal diameter at the diastole (unit: mm) | 0.84 |
| LV internal diameter at the systole (unit: mm) | 0.86 |
| LV posterior wall diameter at the diastole (unit: mm) | 0.86 |
| LV posterior wall diameter at the systole (unit: mm) | 0.88 |
| LA diameter (unit: mm) | 0.91 |
| Aorta diameter (unit: mm) | 0.84 |
| RV diameter (unit: mm) | 0.81 |

The medical indices shown in [Table 1] are examples of measurement values that may be directly obtained from the length of the generated at least one reference line. Additionally, at least one of the medical indices shown in [Table 2] below may be further obtained based on measurement values that may be obtained from the length of at least one reference line.

TABLE 2

| Item | Formula | Description |
|---|---|---|
| EDV | [7/(2.4 + LVIDd]]] × | Internal volume of |
| (End diastolic | LVIDd³ | ventricles at end-diastole of |
| volume) | | heart |
| ESV | [7/(2.4 + LVIDs)]] * | Internal volume of the |
| (End systolic | LVIDs³ | ventricles at end-systole of |
| volume) | | heart |
| EF | (LVIDd² − | the most important and |
| (Ejection | LVIDs²/LVIDd²) × | popular measure of left |
| fraction) | 100(%) | ventricular systolic function. |
| | | It is the ratio of stroke |
| | | volume to end diastolic |
| | | volume (EDV), expressed as |
| | | percentage. |
| LVmass | 0.8 * 1.04 *[(IVS + | Left ventricular mass index |
| | LVID + LVPW)³] + 0.6 | |

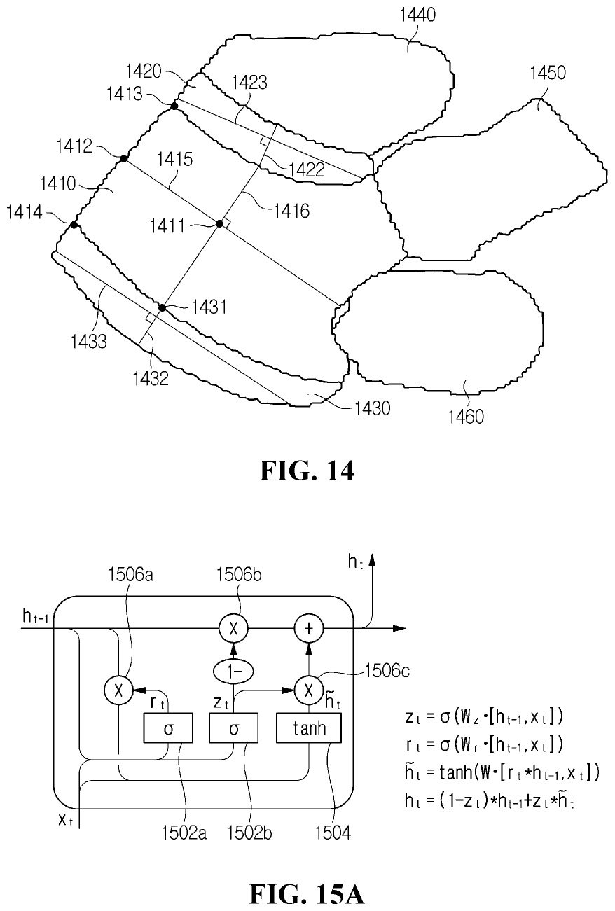

As described above, at least one medical index may be determined from an echocardiography image of the heart. Embodiments for determining medical indices based on the above-described segmentation result, that is, segmentation contours of regions, may also be applied to other types of images. In other words, if segmentation may be performed on a given image, a medical index may be derived based on at least one reference point and at least one reference line. An example of at least one reference point and at least one reference line is shown in FIG. 14.

FIG. 14 illustrates an example of reference points and reference lines determined based on a segmentation result according to an embodiment of the present invention. FIG. 14 illustrates a segmentation result for a given image. Referring to FIG. 14, by segmentation, a first region 1410, a second region 1420, a third region 1430, a fourth region 1440, a fifth region 1450, and a sixth region 1460 are partitioned. The first region 1410 is located between the second region 1420 and the third region 1430, the second region 1420 is located between the fourth region 1440 and the first region 1410, the fourth region 1440 is located at the top, the fifth region 1450 is adjacent to the right side of the first region 1410, and the sixth region 1460 is adjacent to the right side of the first region 1410 and is located at the lower end of the fifth region 1450.

Point a 1411 is the center point of the first region 1410. Here, the center point means central moments in a corresponding region. Point b 1412 is a point that bisects the left boundary line of the first region 1410. Here, one end of the left boundary line is a second left point 1413 where a boundary between the first region 1410 and the second region 1420 is separated at the left side of the first region 1410, and the other end of the left boundary line is a first left region 1414 where a boundary between the first region 1410 and the third region 1430 is separated. A transversal line 1415 passing through point b 1414 and crossing the first region 1410 may be drawn, and an orthogonal line 1416 orthogonal to the transversal line 1415 may be drawn. Here, at least a part of the orthogonal line 1416 may be used as a reference line.

Point c 1421 is a point of contact between the orthogonal line 1416 and the segmentation contour of the second region 1420. An orthogonal line 1423 passing through point c 1421 and orthogonal to the long axis 1423 of the second region 1420 may be drawn, and at least a part of the orthogonal line 1423 may be used as a reference line. That is, a reference line within a corresponding region (e.g., the second region 1420) may be determined in a manner extending from a reference line generated in an adjacent region (e.g., the first region 1410).

Point d 1431 is a point of contact between the orthogonal line 1416 and the segmentation contour of the third region 1430. An orthogonal line 1432 passing through point d 1431 and orthogonal to the long axis 1433 of the third region 1430 may be drawn, and at least a part of the orthogonal line 1432 may be used as a reference line. That is, a reference line within a corresponding region (e.g., the third region 1430) may be determined in a manner extending from a reference line generated in an adjacent region (e.g., the first region 1410).

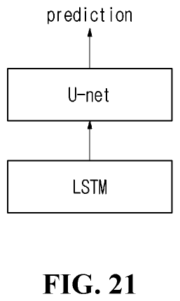

According to various embodiments described above, medical indices may be automatically extracted and provided based on the segmentation result of the medical image. To this end, it is required to perform segmentation on a given medical image. Segmentation of the image is a type of image analysis task, and means an operation of detecting or extracting a region (e.g., a set of pixels or voxels) representing a target of interest among objects expressed in an image. In general, since an image obtained through photographing expresses other objects in addition to the object of interest, it is required to distinguish the object of interest from other objects through image segmentation. Image segmentation may be performed on a single image (e.g., using a model including LSTM and U-net as shown in FIG. 21), but when a time-series image set exists, there is a possibility that the accuracy of image segmentation can be improved by further using several images before or after the image to be segmented. Accordingly, the present invention describes various embodiments of performing image segmentation using a time-series image set. In particular, the present invention describes various embodiments of generating labeled images corresponding to the remaining surrounding images when labels representing a segmentation result are added to only some images of a time-series image set and using the generated labeled images.

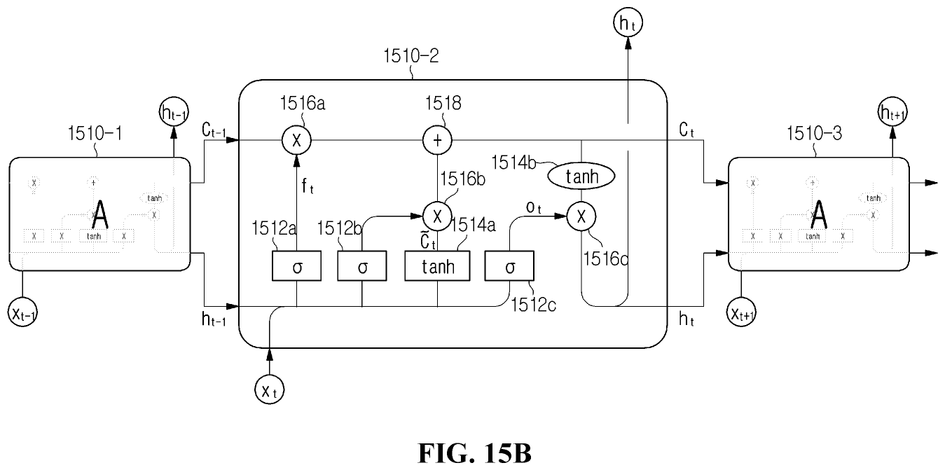

First, as an artificial intelligence model that may be used for segmentation, the structure of a long short-term memory (LSTM) network is as follows. A recurrent neural network (RNN) is an artificial neural network that has a structure of determining a current state by using past input information. The RNN keeps using information, which is obtained in a previous step, by using an iterative structure. As a type of RNN, a long short-term memory (LSTM) network has been proposed. The LSTM network was proposed to control long-term dependency and has an iterative structure like RNN. Th LSTM network has a structure as in FIGS. 15A and 15B.

FIGS. 15A and 15B illustrate an example of an LSTM network applicable to the present invention.

Referring to FIG. 15A, the LSTM network provides a current state h to be transmitted to a next network through h of a previous state (t−1) and input xₜ of a current state (t). The LSTM network calculates the previous state in time series through the state of r, which determines whether to internally use the previous state information. The hidden network includes sigmoid networks 1502a and 502b, a tanh network 1504, multiplication operators 1506a, 506b and 506c, and an addition operator 1508. Each of the sigmoid networks 1502a and 502b has a weight and bias, and uses a sigmoid function as an activation function. The tanh network 1504 has a weight and bias, and uses a sigmoid tanh function as an activation function. The sigmoid network 1502a applies the weighted sum of hₜ₋₁ and xₜ, and then provides a result value rₜ to the multiplication operator 1506a. The sigmoid network 1502b applies the weighted sum of hₜ₋₁ and xₜ, and then provides the result value z t to the multiplication operators 1506b and 506c. The sigmoid network 1502b provides the result value to the multiplication operator 1506b via the 1-operator. The multiplication operator 1506b applies multiplication to hₜ₋₁ and the value provided through the 1-operator from the sigmoid network 1502b and provides it to the addition operator. The tanh network 1504 applies the tanh function to the input xₜ at the current point in time t, and then provides the result value \tilde{h}ₜ to the addition operator through the multiplication operator 1506b. The addition operator applies the input values and outputs hₜ as the result value.

Referring to FIG. 15B, the LSTM network has a structure where hidden networks 1510-1 to 1510-3 are iterated between an input layer and an output layer. Accordingly, when inputs xₜ₋₁, xₜ, xₜ₊₁ and the like are provided over time, a hidden state value, which is output in the hidden network 1510-1 for the input xₜ₋₁ at a time t−1, is input into the hidden network 1510-2 for a next time t together with the input xₜ at the next time t. The hidden network 1510-2 includes sigmoid networks 1512a, 512b and 512c, tanh networks 1514a and 514b, multiplication operators 1516a, 516b and 516c, and an addition operator 1518. Each of the sigmoid networks 1512a, 512b and 512c has a weight and a bias and uses a sigmoid function as an activation function. Each of the tanh networks 1514a and 514b has a weight and a bias and uses a sigmoid tanh function as an activation function.

The sigmoid network 1512a functions as a forget gate. The sigmoid network 1512a applies a sigmoid function to a weighted sum of a hidden state value hₜ₋₁ of a hidden layer of a previous time and input xₜ of a current time and then provides a result value to the multiplication operator 1516a. The multiplication operator 1516a multiplies the result value of the sigmoid function by a cell memory value Cₜ₋₁ of the previous time. Thus, the LSTM network may determine whether or not to forget a memory value of the previous time. That is, an output value of the sigmoid network 1512a indicates how long the cell memory value Cₜ₋₁ of the previous time is to be maintained.

The sigmoid network 1512b and the tanh network 1514 function as an input gate. The sigmoid network 1512b applies a sigmoid function to a weighted sum of a hidden state value hₜ₋₁ of a previous time t−1 and input xₜ of a current time t and then provides a result value iₜ to the multiplication operator 1516b. The tanh network 1514 applies a tanh function to a weighted sum of a hidden state value hₜ₋₁ of a previous time t−1 and input xₜ of a current time t and then provides a result value \tilde{C}ₜ to the multiplication operator 1516b. The result value iₜ of the sigmoid network 1512b and the result value \tilde{C}ₜ of the tanh network 1514 are multiplied by the multiplication operator 1516b and then are provided to the addition operator 1510. Thus, the LSTM network may determine how much the input xₜ of a current time is to be reflected in the cell memory value Cₜ of a current time and then perform scaling according to determination. A cell memory value Cₜ₋₁ of a previous time, which is multiplied by a forget coefficient, and iₜ·\tilde{C}ₜ are added up by the addition operator 1510. Thus, the LSTM network may determine the cell memory value Cₜ of the current time.

The sigmoid network 1512c, the tanh network 1514b, and the multiplication operator 516c function as an output gate. An output gate outputs a filtered value based on a cell state of a current time. The sigmoid network 1512c applies a sigmoid function to a weighted sum of a hidden state value hₜ₋₁ of a previous time t−1 and input xₜ of a current time t and then provides a result value oₜ to the multiplication operator 1516b. The tanh network 1514b applies a tanh function to the cell memory value Cₜ of the current time t and then provides a result value to the multiplication operator 1516c. The multiplication operator 1516c generates a hidden state value hₜ of the current time t by multiplying a result value of the tanh network 514b and a result value of the sigmoid network 1512c. Thus, the LSTM network may determine how long the cell memory value of the current time is to be maintained in a hidden layer.

The LSTM model has various deformation models. FIG. 15B illustrates an example of an LSTM deformation model. The LSTM model used in the present invention is not limited to the above-described embodiment, and various deformation models may be used.

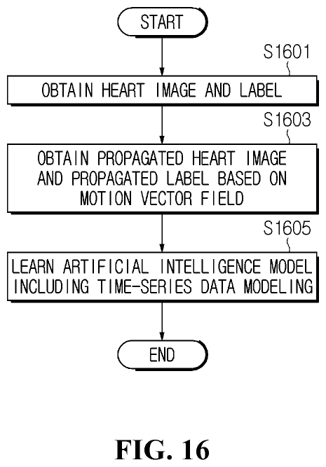

FIG. 16 illustrates an example of a training procedure for segmentation according to an embodiment of the present invention. FIG. 16 illustrates a method of operating a device having computing capability (e.g., the service server 170 of FIG. 1).

Referring to FIG. 16, in step S1601, the device obtains a heart image and a label. The heart image is some of time-series images, and the heart image may have periodicity. The heart image may be a reference image. The label may indicate a boundary of at least one chamber of the heart image. That is, the label may indicate a boundary of at least one of left ventricle, left atrium, right ventricle or right atrium of the heart image. The label may be expressed as annotations, masks, ground truth labels, etc., depending on circumstances.

As another example, the device may obtain an ultrasonic image and label of the lung. The ultrasonic image of the lung is some of time-series images and may have periodicity of expiration and inspiration. The label may indicate at least one boundary of the ultrasonic image of the lung. The present invention may be applied for segmentation of time-series images having periodicity, and is not limited to the above-described embodiment. Lung ultrasound is a diagnostic method with high sensitivity. For example, the device may distinguish pneumonia, atelectasis, tumor, diaphragmatic elevation, and pleural effusion through lung ultrasound, unlike a chest X-ray taken at a patient's bedside. The chest X-ray is routinely performed to evaluate the occurrence of pneumothorax or hydrothorax after procedures such as central vein catheterization. However, the sensitivity of chest X-ray is very low to detect small amounts of pneumothorax or hydrothorax that occur after such a procedure. In contrast, lung ultrasound may enable diagnosis of very small amounts of pneumothorax, consolidation, pulmonary edema, atelectasis, pneumonia, and pleural effusion.

In step S1603, the device may obtain a propagated heart image and a propagated label based on the obtained heart image. The device may obtain the propagated heart image and the propagated label using the heart image obtained in step S1601 and an image having a time-series relationship with the heart image. Specifically, the device may estimate a motion vector field of the heart image obtained in step S1601 and the image having the time-series relationship with the heart image. For example, the device may estimate a motion vector field based on an artificial intelligence model. Here, the device may estimate the motion vector field using a CNN. Alternatively, the device may measure motion based on optical flow. As an example, the device may measure motion based on LiteflowNet. The device may obtain the propagated heart image and the propagated label based on the motion vector field. That is, the device may augment the heart images. Accordingly, the device may augment images included in the cycle based on a reference image, which is a part of images having a cardiac cycle. The device may obtain a dataset sufficient to perform time-series data modeling.

In step S1605, the device may learn an artificial intelligence model including time-series data modeling. Specifically, the device may learn an artificial intelligence model based on the obtained heart image and label, and the propagated heart image and propagated label. For example, the artificial intelligence model may be based on LSTM.

According to the embodiment described with reference to FIG. 16, the device may obtain not only a heart image at a time t, but also a heart image at a time t−1 and an image at a time t+1 close thereto. The device may learn to perform prediction at the time t based on the obtained image. That is, in order to perform prediction on the heart image at the time t, the device may extract features based on the heart image at the time t−1, the heart image at the time t, and the heart image at the time t+1. For example, the device may extract features based on ResNet. Also, the device may learn the extracted features based on LSTM. In addition, the device may use ConvLSTM for time-series heart image modeling. Also, the device may use 3D Cony to model time-series data using adjacent frames. The features extracted from the heart image may be related to a boundary of at least one of a left ventricle, a left atrium, a right ventricle, or a right atrium of the heart image. The device may extract features based on a plurality of images, and is not limited to the number of the above-described embodiments. An artificial intelligence model related to various time-series data modeling may be used, and is not limited to the above-described embodiment.

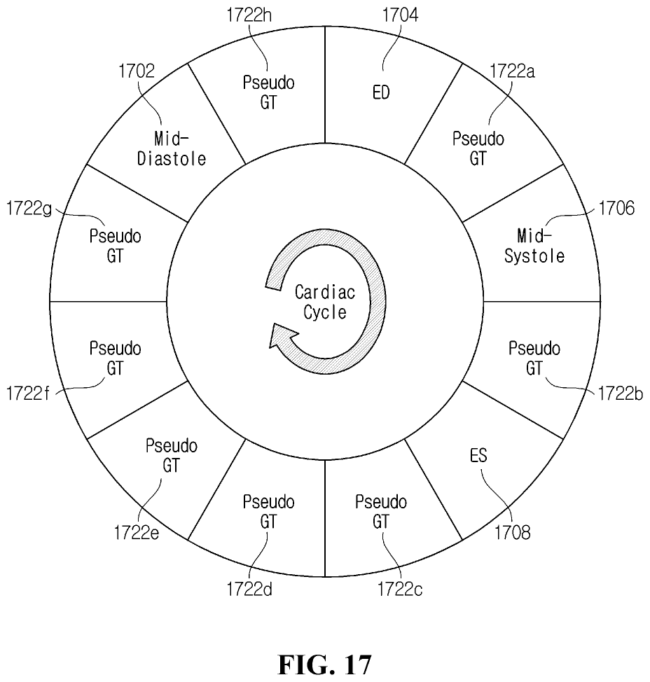

According to the embodiments described with reference to FIG. 16, an image of the heart may be augmented. An example of augmented images is shown in FIG. 17 below. FIG. 17 illustrates a heart image and a propagated heart image according to an embodiment of the present invention. The heart images may have a cardiac cycle. The device may obtain an image of at least one of a mid-diastole 1702, an end-diastole 1704, a mid-systole 1706 or an end-systole 1708 as a reference image among the hart images. That is, the reference image may include an image captured in at least one state of the cardiac cycle, and in this case, the heart state may be determined based on the size of the left ventricle. As a specific example, the device may obtain images of the end-diastole 1704 and the end-systole 1708 as reference images. The reference image refers to an image including a ground truth (GT) label. Images have a time-series relationship with each other and are suitable for use in time-series data modeling. However, it may be insufficient to learn the artificial intelligence of the time-series data modeling structure only when the device obtains 2 to 4 reference images in one cardiac cycle. The device may propagate the image and label from the reference image based on the motion vector field as described above. Accordingly, the device may obtain a plurality of pseudo GTs 1722a to 1722h. The pseudo GTs 1722a to 1722h may constitute a cardiac cycle together with the reference image as a propagated image. When calculating a loss value by a loss function during training of an artificial intelligence model, a weight applied to a prediction result related to the pseudo GTs 1722a to 1722h may be set lower than that of the reference image.

Meanwhile, the pseudo image and the pseudo GT generated based on the motion estimation value of the image may have different shape from the real heart. Accordingly, the weights may be used to update parameter weight values through network learning in real images and real GTs. The generated pseudo image and pseudo GT may be distorted as the motion between the real image and the real GT increases. That is, the larger a time difference within one cardiac cycle, the more difficult it is to estimate the cardiac motion. Accordingly, the weight may decrease as the value of the time difference r with the image serving as the reference for real image creation increases. That is, the weight may decrease as the distance from the reference image increases. Here, the weight may be determined as an experimental value. In addition, in the cardiac cycle, the pulse rate per minute may be 60 to 100, for example, in the case of a normal person. Also, in the case of bradycardia patients with a slow heart rate, the pulse rate per minute may be 60 or less. In addition, in the case of tachycardia patients with fast heart rate, the pulse rate per minute may be 100 or more.

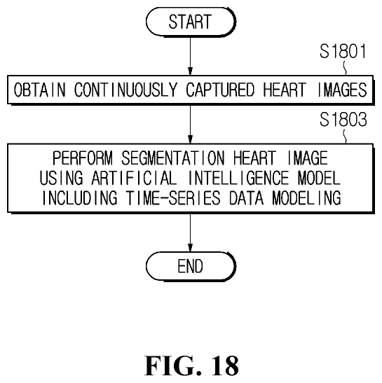

FIG. 18 illustrates an example of a segmentation procedure according to an embodiment of the present invention. FIG. 18 illustrates a method of operating a device having computing capability (e.g., the service server 170 of FIG. 1).

Referring to FIG. 18, in step S1801, the device obtains continuously captured heart images. That is, the device may obtain a plurality of heart images. The plurality of obtained images may have a time-series relationship. For example, heart images may be provided in real time during imaging by ultrasound equipment.

In step S1803, the device may perform segmentation on the obtained heart image using an artificial intelligence model including time-series data modeling. According to an embodiment, the device uses time-series images (e.g., n images from a time t to a time t+n−1) as input data, and generate a segmentation result of images at one of the time t to the time t+n−1, a time after the time t+n−1 or a time before the time t. Here, the artificial intelligence model may be in a trained state based on the propagated heart image, the propagated label, and the weight as in the procedure described with reference to FIG. 16.

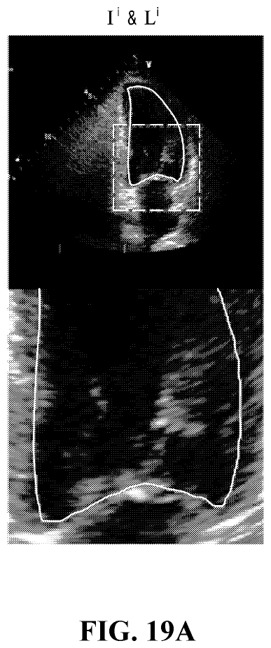

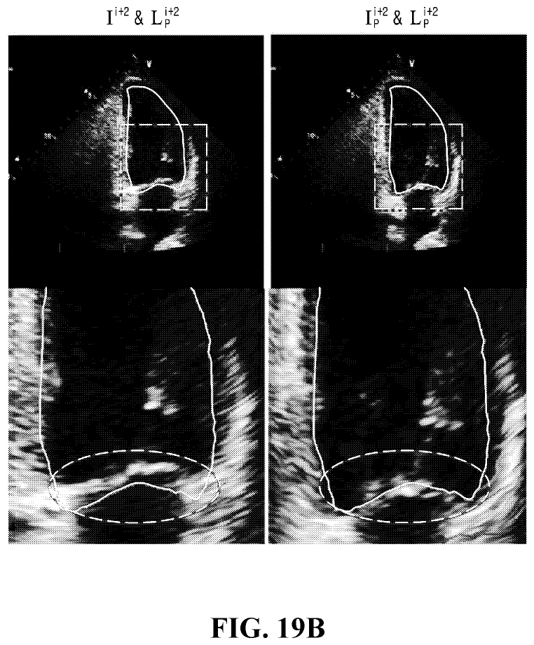

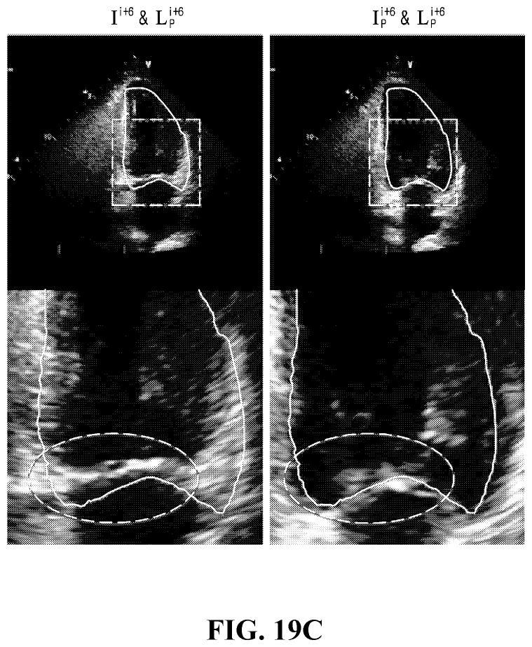

FIGS. 19A, 19B, and 19C illustrate specific examples of data augmentation according to an embodiment of the present invention. Specifically, FIG. 19 illustrates an example of an A2CH echocardiography view. FIG. 19A illustrates an original image and annotation mask. FIG. 19B illustrates an original image and a propagated mask. FIG. 19C illustrates a propagated image and a propagated mask. The upper part of each picture shows the visualization of image and mask. The lower part of each picture represents the magnified view, and the green line represents the boundary of LV. A label and a mask may be used interchangeably.

Heart images are 2D images that change over time. To efficiently augment heart images and annotated masks, the device may measure a cardiac motion vector field and propagate reference data. Here, the term propagate may indicate an example of an image conversion method, unlike propagation on a neural network. For example, the device may measure the cardiac motion vector field based on a convolutional neural network (CNN).

The device may generate pairwise data composed of a label and an image. For example, the device may generate pairwise data composed of an image without a propagated label and a ground truth label. The device may augment this data. An image without a ground true label at a time point separated by m (frame) from the reference image may be expressed as I^{i+m}. The m-th augmented propagated label may be expressed as L_{P}^{i+m}.

The boundary between an image without an augmented ground true label and an augmented propagated label may not match. For example, the boundary between an image without an augmented label and an augmented propagated label may not match based on erroneousness of a motion vector field. In addition, as a propagation step increases, the boundary error between the image without the augmented label and the augmented propagated label may increase. Referring to FIG. 19B, when the propagation step size of a non-propagated image and a propagated label is 2, an error exists between the image and the label. Referring to FIG. 19C, it can be seen that an error exists when the propagation step size of the non-propagated image and the propagated label is 6, and the error is larger than that when the step size is 2. Sequential propagation of estimating a motion vector field using a past image and a current image may cause an error in the motion vector field and may distort the original image.

The device may reduce the error of the motion vector field by propagating the image as well as the label. In addition, the device may augment accurate data by making the propagation step of the label equal to the propagation step of the image.

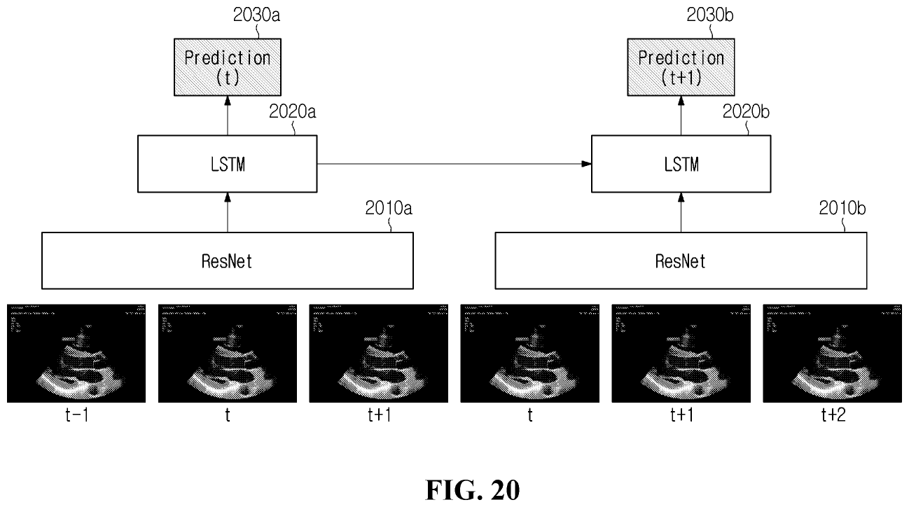

FIG. 20d illustrates a specific implementation example of an artificial intelligence model for segmentation according to an embodiment of the present invention. Referring to FIG. 20, the artificial intelligence model includes ResNets 2010a and 2010b and LSTMs 2020a and 2020b. In FIG. 20, ResNets 2010a and 2010b are represented by a plurality of networks, but this expresses that the feature extraction operation by ResNets 2010a and 2010b is performed for each time point, and the artificial intelligence model may include one ResNet. Similarly, LSTMs 2020a and 2020b are represented by a plurality of networks, but the artificial intelligence model may include one LSTM having a recursive structure in which an output is fed back to an input.

Referring to FIG. 20, the device performs prediction on the image at a time t using not only the image at the time t, but also the image at a time t−1 and the image at a time t+1 close thereto. The ResNet 2010a may extract features of the image at the time t−1, the image at the time t, and the image at the time t+1. The extracted features are input to the LSTM 2020a for implementing time-series data modeling. The LSTM 2020a generates a prediction result 2030a based on the features, and delivers a hidden state corresponding to the prediction result 2030a to the LSTM 2020b. The ResNet 2010b extracts the features of the image at the time t, the image at the time t+1, and the image at the time t+2, and the LSTM 2020b generates a prediction result 2030b based on the extracted features and the hidden state provided from the LSTM 2020a.

The exemplary methods of the present invention are represented in a series of operations for clarity of description, but this is not intended to limit the order in which the steps are performed, and each step may be performed simultaneously or in a different order, if necessary. In order to realize a method according to the present invention, the steps illustrated may include further other steps, or may include the remaining steps with the exception of some steps, or may include additional other steps with the exception of some steps.

Various embodiments of the present invention are not intended to enumerate all possible combinations, but to describe a representative aspect of the present invention, and the matters described in the various embodiments may be applied independently or in combination of two or more.

In addition, various embodiments of the present invention may be realized by hardware, firmware, software, or a combination thereof. In the case of hardware realization, the embodiments may be realized by one or more ASICs (Application Specific Integrated Circuits), DSPs (Digital Signal Processors), DSPDs (Digital Signal Processing Digital Signal Processing Devices (DSPs), Programmable Logic Devices (PLDs), Field Programmable Gate Arrays (FPGAs), general processors, controllers, microcontrollers, microprocessors, etc.

The scope of the present invention includes software or machine-executable commands (e.g., operating systems, applications, firmware, programs, etc.) that allow an operation according to a method of various embodiments to be performed on a device or computer, and a non-transitory computer-readable medium in which such software or commands are stored and executed on the device or computer.

1. A method of obtaining information from a medical image, the method comprising:
performing segmentation on regions of a heart in the medical image;
generating at least one reference line based on the segmentation; and
determining at least one medical index based on the at least one reference line.

2. The method of claim 1, wherein the at least one medical index comprises at least one of a length of the at least one reference line or a value determined based on the length of the at least one reference line.

3. The method of claim 1, further comprising generating at least one reference point based on the segmentation.

4. The method of claim 3, wherein the at least one reference point is generated based on at least one center point of the regions.

5. The method of claim 1, wherein the regions comprise at least one of a first region, a second region, a third region, a fourth region, a fifth region or a sixth region.

6. The method of claim 5, wherein:
the first region is located between the second region and the third region,
the second region is located between the fourth region and the first region,
the fourth region is located at the top,
the fifth region is adjacent to a right side of the first region, or
the sixth region is adjacent to the right side of the first region and is located below the fifth region.

7. The method of claim 5, wherein the generating the at least one reference line comprises:
identifying a transversal line passing through a reference point of the first region and a center of a left boundary line of the first region and crossing the first region;
identifying a first orthogonal line passing through the reference line and orthogonal to the transversal line; and
generating a first reference line to include at least a part of the first orthogonal line.

8. The method of claim 7, wherein the generating the at least one reference line comprises:
identifying a second orthogonal line passing through a point of contact between the first reference line and a segmentation contour of the second region and orthogonal to a long axis of the second region; and
generating a second reference line to include at least a part of the second orthogonal line.

9. The method of claim 7, wherein the generating the at least one reference line comprises:
identifying a third orthogonal line passing through a point of contact between the first reference line and a segmentation contour of the third region and orthogonal to a long axis of the third region; and
generating a third reference line to include at least a part of the third orthogonal line.

10. The method of claim 7, wherein the generating the at least one reference line comprises:
identifying a center line of a short axis of a region corresponding to the second region;
identifying a first vertical line perpendicular to a boundary line of the second region at a point where the center line and the boundary line of the region meet; and
generating a first reference line to include at least a part of the first vertical line, and
wherein the first reference line is used to measure a diameter of the first region.

11. The method of claim 7, wherein the generating the at least one reference line comprises:
identifying a second vertical line perpendicular to a boundary line of the third region at a point where the first reference line and the boundary line of the third region meet; and
generating a second reference line to include at least a part of the second vertical line, and
wherein the second reference line is used to measure a thickness of the third region.

12. The method of claim 5, wherein the generating the at least one reference line comprises:
identifying a closest point closest to the fourth region among points on a boundary line of the fifth region;
identifying a parallel line including the closest point and parallel to a junction of the first region and the fifth region; and
generating a third reference line to include at least a part of the parallel line.

13. The method of claim 5, wherein the generating the at least one reference line comprises:
identifying a sinus point on a boundary line of the fifth region;
identifying a vertical line including a point closest to the sinus point on a boundary line of a region corresponding to the sixth region, parallel to a vertical axis of the medial image and penetrating the inside of a region corresponding to the sixth region; and
generating a fourth reference line to include at least a part of the vertical line.

14. The method of claim 5, wherein the generating the at least one reference line comprises:
identifying a sinus point on a boundary line of the fifth region;
identifying a vertical line including a point closest to the sinus point on a boundary line of a region corresponding to the sixth region, perpendicular to a long axis of a region corresponding to the sixth region and penetrating the inside of the region corresponding to the sixth region; and
determining a fourth reference line to include at least a part of the vertical line.

15. The method of claim 5, wherein the generating the at least one reference line comprises:
identifying a vertical line including one point on a junction of the fifth region and the first region and perpendicular to a center line of a long axis of the fourth region; and
generating a fifth reference line to include at least a part of the vertical line.

16. The method of claim 5, wherein the generating the at least one reference line comprises:
identifying a vertical line including one point on a junction of the fifth region and the first region and perpendicular to a vertical axis of the medical image; and
generating a fifth reference line to include at least a part of the vertical line.

17. The method of claim 1, wherein the regions comprise at least one of right ventricle (RV), interventricular septum (IVS), aorta, left ventricle (LV), LV posterior wall (LVPW) or left atrium (LA).

18. A device for obtaining information from a medical image, the device comprising:
a storage unit configured to store a set of commands for operation of the device; and
at least one processor connected to the storage unit,
wherein the at least one processor is configured to:
perform segmentation on regions of a heart in the medical image;
generate at least one reference line based on the segmentation; and
determine at least one medical index based on the at least one reference line.

19. (canceled)

20. A non-transitory computer-readable medium storing at least one instruction, comprising the at least one instruction that is executable by a processor,
wherein the at least one instruction controls a device for obtaining information from a medical image to:
perform segmentation on regions of a heart in the medical image;
generate at least one reference line based on the segmentation; and
determine at least one medical index based on the at least one reference line.
